SEQUENCE LISTING (6 sequences)

SEQ ID NO: 1 - protein, 251 aa, Pseudomonas stutzeri
MRTHFTALLLLFSLSSLANDEVPELRIEKVKENIFLHTSYSRVNGFGLVSSNGLVVIDKGNAFIVDTPWSDRDTETLVHWIRKNGYELLGSVSTHWHEDRTAGIKWLNDQSISTYATTSTNHLLKENKKEPAKYTLKGNESTLVDGLIEVFYPGGGHTIDNVVVWLPKSKILFGGCFVRSLDSEGLGYTGEAHIDQWSRSAQNALSRYSEAQIVIPGHGKIGDIALLKHTKSLAETASNKSIQPNANASAD

SEQ ID NO: 2 - DNA, 822 bp, Pseudomonas stutzeri
atgagaacacattttacagcgttattacttctattcagcttgtcttcgcttgctaacgacgaggtacctgagctaagaatcgagaaagtaaaagagaacatctttttgcacacatcatacagtcgtgtgaatgggtttggtttggtcagttcaaacggccttgttgtcatagataagggtaatgctttcattgttgatacaccttggtcagaccgagatacagaaacgctcgtacattggattcgtaaaaatggttatgagctactggggagtgtttctactcattggcatgaggatagaaccgcaggaattaaatggcttaatgaccaatcaatttctacgtatgccacgacttcaaccaaccatctcttgaaagaaaataaaaaagagccagcgaaatacaccttgaaaggaaatgagtccacattggttgacggccttatcgaagtattttatccaggaggtggtcatacaatagacaacgtagtggtgtggttgccaaagtcgaaaatcttatttggcggctgttttgtgcgtagccttgattccgaggggttaggctacactggtgaagcccatattgatcaatggtcccgatcagctcagaatgctctgtctaggtactcagaagcccagatagtaattcctggccatgggaaaatcggggatatagcgctgttaaaacacaccaaaagtctggctgagacagcctctaacaaatcaatccagccgaacgctaacgcgtcggctgattgaggcgttaggccgcatggacacaacgcaggtcacattgatacacaaaattctagctgcggcagatga

SEQ ID NO: 3 - DNA, 20 bp, Artificial
  primer
tctattcagcttgtcttcgc

SEQ ID NO: 4 - DNA, 20 bp, Artificial
  primers
tgttagaggctgtctcagcc

SEQ ID NO: 5 - DNA, 7 bp, Pseudomonas stutzeri
gttagag

SEQ ID NO: 6 - DNA, 7 bp, Pseudomonas stutzeri
cgctaac

(19) United States
(12) Patent Application Publication
(10) Pub. No.: US 2012/0219952 A1
(43) Pub. Date: Aug. 30, 2012

(54) CARBAPENEMASE AND ANTIBACTERIAL TREATMENT

(76) Inventors: Patrice Nordmann, Le Kremlin Bicetre (FR); Laurent Poirel, Le Kremlin Bicetre (FR)

(21) Appl. No.: 13/500,665

(22) PCT Filed: Oct. 6, 2010
(86) PCT No.: PCT/EP2010/064886

§ 371 Date: May 4, 2012

(30) Foreign Application Priority Data
Oct. 6, 2009 (EP) 09305946.7

(51) Int. Cl. C12Q 1/68; G01N 33/53; C12Q 1/34; C12N 9/86; C07H 21/04

(52) U.S. Cl. 435/6.11; 435/231; 536/23.2; 435/18; 435/7.8; 435/6.15

(57) ABSTRACT

The present invention relates to a carbapenemase and methods using said carbapenemase such as screening methods, predictive methods and therapeutic uses.

### FIELD OF THE INVENTION

The present invention relates to carbapenemases and methods using said carbapenemases such as screening methods, predictive methods and therapeutic uses.

### BACKGROUND OF THE INVENTION

The discovery and the development of antibacterial compounds has been a real progress in medicine and permitted to save many lives. But the development of antibacterial resistances became a real public health issue.

Today, the understanding of resistance mechanisms and the development of new drugs able to bypass the resistance mechanisms constitute a way of research for the progress in new strategies of treatment for infectious diseases.

Particularly, a class of enzyme called carbapenemases are responsible of mechanism of resistance against β-lactams by hydrolyze of the β-lactam ring of this antibiotic class. Production of these carbapenemases among Gram negatives currently represents one of the most challenging traits in antibiotic resistance. Currently, there are several carbapenemases described but the discovery of new members of this enzyme family permits to develop new strategies of diagnosis of emerging antibiotic resistance determinants.

### SUMMARY OF THE INVENTION

The present invention relates to a carbapenemase comprising or consisting of the amino acid sequence defined by SEQ ID NO:1 and a nucleic acid sequence encoding said carbapenemase.

The invention also relates to a method for screening an antibacterial substance comprising the step of determining the ability of a candidate substance to inhibit the activity of a purified carbapenemase of the invention.

The invention further relates to a method for screening an antibacterial substance, wherein said method comprises the steps of:
(i) providing a candidate substance;
(ii) assaying said candidate substance for its ability to bind to a carbapenemase of the invention;

The invention also provides a method for screening an antibacterial substance, wherein said method comprises the steps of:
(i) contacting a candidate substance with a carbapenemase of the invention;
(ii) detecting the complexes eventually formed between said carbapenemase and said candidate substance.

The invention relates to a method for detecting or predicting a resistance mechanism of a microorganism against β-lactams comprising the step of assaying the presence or the expression of a gene encoding a carbapenemase of the invention in said microorganism.

The invention also relates to a method for predicting the response to an antibacterial treatment containing a β-lactam compound and an inhibitor of a carbapenemase of the invention in a patient, comprising the step of determining if the microorganism responsible for the infection in said patient expresses said carbapenemase.

The invention further relates to a method for predicting the response to an antibacterial treatment using aztreonam in a patient comprising the step of determining if the microorganism responsible for the infection in said patient expresses a carbapenemase of the invention.

### DETAILED DESCRIPTION OF THE INVENTION

### Definitions

By “purified” and “isolated” it is meant, when referring to a polypeptide or a nucleotide sequence, that the indicated molecule is present in the substantial absence of other biological macromolecules of the same type. The term “purified” as used herein preferably means at least 75% by weight, more preferably at least 85% by weight, more preferably still at least 95% by weight, and most preferably at least 98% by weight, of biological macromolecules of the same type are present. An “isolated” nucleic acid molecule which encodes a particular polypeptide refers to a nucleic acid molecule which is substantially free of other nucleic acid molecules that do not encode the subject polypeptide; however, the molecule may include some additional bases or moieties which do not deleteriously affect the basic characteristics of the composition.

Two amino acid sequences are “substantially homologous” or “substantially similar” when greater than 80%, preferably greater than 85%, preferably greater than 90% of the amino acids are identical, or greater than about 90%, preferably grater than 95%, are similar (functionally identical). Preferably, the similar or homologous sequences are identified by alignment using, for example, the GCG (Genetics Computer Group, Program Manual for the GCG Package, Version 7, Madison, Wis.) pileup program, or any of sequence comparison algorithms such as BLAST, FASTA, etc.

As used herein, the term “subject” refers to a human or another mammal (e.g., primate, dog, cat, goat, horse, pig, mouse, rat, rabbit, and the like), that can be infected with a strain. In a particular embodiment of the present invention, the subject is a human. In particular, the subject can be a patient.

In its broadest meaning, the term “treating” or “treatment” refers to reversing, alleviating, inhibiting the progress of the disorder or condition to which such term applies, or one or more symptoms of such disorder or condition.

“Pharmaceutically” or “pharmaceutically acceptable” refer to molecular entities and compositions that do not produce an adverse, allergic or other untoward reaction when administered to a mammal, especially human, as appropriate.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The term “β-lactam” has its general meaning in the art and refers to a broad class of antibiotics that include penicillin derivatives, cephalosporins, monobactams, carbapenems, and β-lactam molecules action as β-lactamase inhibitors. Said family of antibiotics is characterised by a β-lactam nucleus (see the formula below) in its molecular structure:

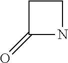

β-lactam compounds include, but are not limited to, imipenem, meropenem, ertapenem, faropenem, doripenem and panipenem.

The term “carbapenemase” has its general meaning in the art and refers to a class of enzymes produced by some bacteria belonging to the β-lactamase family. Said enzymes may be responsible for resistance to β-lactam antibiotics like oxyiminocephalosporins, cephamycins and carbapenems by hydrolyzing β-lactam cycle of said antibiotics.

### Enzymes and Nucleic Acids of the Invention

The inventors have identified a carbapenemase herein after named DIM-1 which hydrolyses all β-lactams except aztreonam.

Thus, a first object of the invention relates to a carbapenemase comprising or consisting of the amino acid sequence defined by SEQ ID NO:1.

In another embodiment, the invention relates to a carbapenemase having at least 80% amino acid sequence identity with the amino acid sequence of SEQ ID NO:1, preferably at least 85% amino acid sequence identity with the amino acid sequence of SEQ ID NO:1 and more preferably having at least 90% amino acid sequence identity with the amino acid sequence of SEQ ID NO:1.

TABLE 1
Amino acid sequence of the premature protein
DIM-1 (SEQ ID NO: 1).

MRTHF TALLL LFSLS SLAND EVPEL RIEKV KENIF LHTSY
SRVNG FGLVS SNGLV VIDKG NAFIV DTPWS DRDTE TLVHW
IRKNG YELLG SVSTH WHEDR TAGIK WLNDQ SISTY ATTST
NHLLK ENKKE PAKYT LKGNE STLVD GLIEV FYPGG GHTID
NVVVW LPKSK ILFGG CFVRS LDSEG LGYTG EAHID QWSRS
AQNAL SRYSE AQIVI PGHGK IGDIA LLKHT KSLAE TASNK
SIQPN ANASA D

A further object of the invention relates to a nucleic acid sequence encoding a carbapenemase of the invention.

In a particular embodiment, the invention relates to a nucleic acid sequence encoding the DIM-1 carbapenemase defined by SEQ ID NO:2.

TABLE 2
Nucleic acid sequence of DIM-1 (SEQ ID NO: 2).

ATG AGA ACA CAT TTT ACA GCG TTA TTA CTT CTA TTC AGC
TTG TCT TCG CTT GCT AAC GAC GAG GTA CCT GAG CTA AGA
ATC GAG AAA GTA AAA GAG AAC ATC TTT TTG CAC ACA TCA
TAC AGT CGT GTG AAT GGG TTT GGT TTG GTC AGT TCA AAC
GGC CTT GTT GTC ATA GAT AAG GGT AAT GCT TTC ATT GTT
GAT ACA CCT TGG TCA GAC CGA GAT ACA GAA ACG CTC GTA
CAT TGG ATT CGT AAA AAT GGT TAT GAG CTA CTG GGG AGT
GTT TCT ACT CAT TGG CAT GAG GAT AGA ACC GCA GGA ATT
AAA TGG CTT AAT GAC CAA TCA ATT TCT ACG TAT GCC ACG
ACT TCA ACC AAC CAT CTC TTG AAA GAA AAT AAA AAA GAG
CCA GCG AAA TAC ACC TTG AAA GGA AAT GAG TCC ACA TTG
GTT GAC GGC CTT ATC GAA GTA TTT TAT CCA GGA GGT GGT
CAT ACA ATA GAC AAC GTA GTG GTG TGG TTG CCA AAG TCG
AAA ATC TTA TTT GGC GGC TGT TTT GTG CGT AGC CTT GAT
TCC GAG GGG TTA GGC TAC ACT GGT GAA GCC CAT ATT GAT
CAA TGG TCC CGA TCA GCT CAG AAT GCT CTG TCT AGG TAC
TCA GAA GCC CAG ATA GTA ATT CCT GGC CAT GGG AAA ATC
GGG GAT ATA GCG CTG TTA AAA CAC ACC AAA AGT CTG GCT
GAG ACA GCC TCT AAC AAA TCA ATC CAG CCG AAC GCT AAC
GCG TCG GCT GAT TGA GGC GTT AGG CCG CAT GGA CAC AAC
GCA GGT CAC ATT GAT ACA CAA AAT TCT AGC TGC GGC AGA
TGA

A carbapenemase of the invention can be produced as a recombinant protein.

For obtaining a recombinant form of a carbapenemase of the invention, or a biologically active fragment thereof, the one skilled in the art may insert the nucleic acid encoding the corresponding polypeptide (SEQ ID NO:2), e.g. into a suitable expression vector and then transform appropriate cells with the resulting recombinant vector. Methods of genetic engineering for producing the polypeptides having a carbapenemase activity according to the invention under the form of recombinant polypeptides are well known from the one skilled in the art.

As it is well known from the one skilled in the art, the recombinant vector preferably contains a nucleic acid that enables the vector to replicate in one or more selected host cells.

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

Expression and cloning vectors usually contain a promoter operably linked to the nucleic acid sequence encoding the polypeptide of interest to direct mRNA synthesis. Promoters recognized by a variety of potential host cells are well known. Promoters suitable for use with prokaryotic hosts include the β-lactamase and lactose promoter systems (Chang et al., 1978; Goeddel et al., 1979), alkaline phosphatase, a tryptophan (trp) promoter system (Goeddel, 1980; EP 36,776), and hybrid promoters such as the tac promoter (deBoer et al., 1983). Promoters for use in bacterial systems also will contain a Shine-Dalgarno (S.D.) sequence operably linked to the DNA encoding the polypeptide of interest.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding polypeptide of interest.

Illustratively, a recombinant vector having inserted therein a nucleic acid encoding a polypeptide of interest according to the invention having a carbapenemase activity may be transfected to bacterial cells in view of the recombinant polypeptide production, e.g. E. coli cells as shown in the examples herein.

Then, the recombinant polypeptide of interest having a carbapenemase activity may be purified, e.g. by one or more chromatography steps, including chromatography steps selected from the group consisting of affinity chromatography, ion exchange chromatography and size exclusion chromatography.

Illustratively, the recombinant polypeptide of interest having a carbapenemase activity may be purified by performing a purification method comprises (a) a step of affinity chromatography, e.g. on a Ni2+-nitriloacetate-agarose resin, (b) a step of anion exchange chromatography with the eluate of step (a) and (c) a size exclusion chromatography with the eluate of step (b).

The purified recombinant polypeptide of interest having a carbapenemase activity may be subjected to a concentration step, e.g. by ultrafiltration, before being stored in an appropriate liquid solution, e.g. at a temperature of −20° C.

Alternatively, a recombinant polypeptide of interest having a carbapenemase activity may be produced by known methods of peptide synthesis. For instance, the polypeptide sequence of interest, or portions thereof, may be produced by direct peptide synthesis using solid-phase techniques. (See, e.g., Stewart et al., 1969; Merrifield, 1963). In vitro protein synthesis may be performed using manual techniques or by automation. Automated synthesis may be accomplished, for instance, with an Applied Biosystems Peptide Synthesizer (Foster City, Calif.) using manufacturer's instructions. Various portions of the polypeptide of interest may be chemically synthesized separately and combined using chemical or enzymatic methods to produce the full-length polypeptide of interest.

Methods of Screening

A further object of the invention relates to a method for screening an antibacterial substance comprising the step of determining the ability of a candidate substance to inhibit the activity of a purified carbapenemase of the invention.

In a particular embodiment, said method comprises the steps of:
(i) providing a composition comprising a carbapenemase of the invention and a substrate thereof,
(ii) adding the candidate substance to be tested to the composition provided at step (i), whereby providing a step composition; and
(iii) comparing the activity of said carbapenemase in the said test composition with the activity of said carbapenemase in the absence of said candidate substance;
(iv) selecting positively the said candidate substance that inhibits the catalytic activity of said carbapenemase.

As intended herein, a candidate substance to be tested inhibits the catalytic activity of said carbapenemase if the activity of the said enzyme, when the candidate is present, is lower than when the said enzyme is used without the candidate substance under testing.

Preferably, the candidate substances that are positively selected at step (iv) of the method above are those that cause a decrease of the hydrolyze of the beta-lactam cycle of β-lactams that leads to less than 0.5 times the hydrolyze rate of the same enzyme in the absence of the candidate substance, more preferably a decrease that leads to less 0.3, 0.2, 0.1, 0.05 or 0.025 times the hydrolyze rate of the same enzyme in the absence of the candidate substance. The most active candidate substances that may be positively selected at step (iv) of the method above may completely block the catalytic activity of said enzyme, which leads to an hydolyze rate of beta-lactam cycle which is undetectable, i.e. zero, or very close to zero.

In a particular embodiment of the screening method described above, the catalytic activity of the carbapenemase of the invention is assessed using as a substrate a molecule of the class of β-lactams except aztreonam. Preferably, said molecule is selected from the group of ticarcillin, piperacillin-tazobactam, imipenem, meropenem, ceftazidime and cefepime and more preferably from the group of ticarcillin, piperacillin-tazobactam, imipenem and meropenem.

Accordingly, the catalytic activity of said carbapenemase is determined by detecting or quantifying the formation of a derivative of β-lactam molecule that results from the opening β-lactam ring as determined by detection of this opened derivative by UV spectrophotometry.

As detailed previously in the specification, this invention encompasses methods for the screening of candidate antibacterial substances that inhibit the activity of a carbapenemase as defined herein.

However, this invention also encompasses methods for the screening of candidate antibacterial substances that are based on the ability of said candidate substances to bind to a carbapenemase as defined herein, thus methods for the screening of potentially antibacterial substances.

The binding assays can be performed in a variety of formats, including protein-protein binding assays, biochemical screening assays, immunoassays, and cell-based assays, which are well characterized in the art.

All binding assays for the screening of candidate antibacterial substances are common in that they comprise a step of contacting the candidate substance with a carbapenemase as defined herein, under conditions and for a time sufficient to allow these two components to interact.

These screening methods also comprise a step of detecting the formation of complexes between said carbapenemase and said candidate antibacterial substances.

Thus, screening for antibacterial substances includes the use of two partners, through measuring the binding between two partners, respectively a carbapenemase as defined herein and the candidate compound.

In binding assays, the interaction is binding and the complex formed between a carbapenemase as defined above and the candidate substance that is tested can be isolated or detected in the reaction mixture. In a particular embodiment, the carbapenemase as defined above or alternatively the antibacterial candidate substance is immobilized on a solid phase, e.g., on a microtiter plate, by covalent or non-covalent attachments. Non-covalent attachment generally is accomplished by coating the solid surface with a solution of the carbapenemase of the invention and drying. Alternatively, an immobilized antibody, e.g., a monoclonal antibody, specific for the carbapenemase of the invention to be immobilized can be used to anchor it to a solid surface. The assay is performed by adding the non-immobilized component, which may be labeled by a detectable label, to the immobilized component, e.g., the coated surface containing the anchored component. When the reaction is complete, the non-reacted components are removed, e.g., by washing, and complexes anchored on the solid surface are detected. When the originally non-immobilized component carries a detectable label, the detection of label immobilized on the surface indicates that complexing occurred. Where the originally non-immobilized component does not carry a label, complexing can be detected, for example, by using a labeled antibody specifically binding the immobilized complex.

The binding of the antibacterial candidate substance to a carbapenemase of the invention may be performed through various assays, including traditional approaches, such as, e.g., cross-linking, co-immunoprecipitation, and co-purification through gradients or chromatographic columns. In addition, protein-protein interactions can be monitored by using a yeast-based genetic system described by Fields and co-workers (Fields and Song, 1989; Chien et al., 1991) as disclosed by Chevray and Nathans, 1991. Many transcriptional activators, such as yeast GAL4, consist of two physically discrete modular domains, one acting as the DNA-binding domain, the other one functioning as the transcription-activation domain. The yeast expression system described in the foregoing publications (generally referred to as the “two-hybrid system”) takes advantage of this property, and employs two hybrid proteins, one in which the target protein is fused to the DNA-binding domain of GAL4, and another, in which candidate activating proteins are fused to the activation domain. The expression of a GAL1-lacZ reporter gene under control of a GAL4-activated promoter depends on reconstitution of GAL4 activity via protein-protein interaction. Colonies containing interacting polypeptides are detected with a chromogenic substrate for .beta.-galactosidase. A complete kit (MATCHMAKER™) for identifying protein-protein interactions between two specific proteins using the two-hybrid technique is commercially available from Clontech. This system can also be extended to map protein domains involved in specific protein interactions as well as to pinpoint amino acid residues that are crucial for these interactions.

Thus, another object of the invention consists of a method for the screening of antibacterial substances, wherein said method comprises the steps of:
(i) providing a candidate substance;
(ii) assaying said candidate substance for its ability to bind to a carbapenemase of the invention;

The same method may also be defined as a method for the screening of antibacterial substances, wherein said method comprises the steps of:
(i) contacting a candidate substance with a carbapenemase of the invention;
(ii) detecting the complexes eventually formed between said carbapenemase and said candidate substance.

The candidate substances, which may be screened according to the screening method above, may be of any kind, including, without being limited to, natural or synthetic compounds or molecules of biological origin such as polypeptides.

### Assessment of the Ex Vivo and in Vivo Activity of the Inhibitors Selected by the Screening Methods of the Invention

Inhibitor substances positively selected at the end of the in vitro screening methods as described above are inhibitors of a carbapenemase of the invention. Accordingly, the activity of selected candidate can be studied by assaying the antibacterial activity of a combination of such compounds with a β-lactam compound against gram negative bacteria expressing a carbapenemase of the invention.

Particularly, the β-lactam compounds which can be used in combination with said inhibitor substances are β-lactams which are hydrolyzed by the carbapenemases of the invention such as ticarcillin, piperacillin-tazobactam, imipenem, meropenem, ertapenem, ceftazidime and cefepime.

An example of bacterial strain expressing a carbapenemase of the invention is Pseudomonas stutzeri. Thus, the antibacterial activity of a combination of an inhibitor substance with a β-lactam compound can be tested against this Gram-negative bacterial strain.

Inhibitor substances that have been positively selected at the end of any one of the in vitro screening methods of the invention may then be assayed for their ex vivo activity in combination with a β-lactam compound, in a further stage of their selection as a useful antibacterial active ingredient of a pharmaceutical composition.

By “ex vivo” antibacterial activity, it is intended herein the antibacterial activity of the combination of a positively selected candidate compound and a β-lactam compound against bacterial cells expressing a carbapenemase of the invention that are cultured in vitro.

Thus, any substance that has been shown to behave like an inhibitor of a carbapenemase, after positive selection at the end of any one of the in vitro screening methods that are disclosed previously in the present specification, may be further assayed for his ex vivo antibacterial activity against bacterial cells expressing a carbapenemase of the invention.

Consequently, any one of the screening methods that are described above may comprise a further step of assaying a combination with a positively selected inhibitor substance and a β-lactam compound for its ex vivo antibacterial activity.

Usually, said further step consists of preparing in vitro cultures of bacterial cells expressing a carbapenemase of the invention and then adding to said bacterial cultures the combination to be tested, before determining the ability of said candidate compound to block bacterial growth or even most preferably kill the cultured bacterial cells.

Typically, bacterial cells are plated in Petri dishes containing the appropriate culture medium, generally in agar gel, at a cell number ranging from 10 to 10³ bacterial cells, including from 10 to 10² bacterial cells. In certain embodiments, serials of bacterial cultures are prepared with increasing numbers of seeded bacterial cells.

Typically, the combination to be tested is then added to the bacterial cultures, preferably with a serial of amounts of said candidate compounds for each series of a given plated cell number of bacterial cultures.

Then, the bacterial cultures are incubated in the appropriate culture conditions, most preferably starvation conditions, for instance in a cell incubator at the appropriate temperature, and for an appropriate time period, for instance a culture time period ranging from 1 day to 4 days, before counting the resulting CFUs (Colony Forming Units), either manually under a light microscope or binocular lenses, or atomically using an appropriate apparatus.

Generally, appropriate control cultures are simultaneously performed i.e; negative control cultures without the combination and positive control cultures with an antibiotic that is known to be toxic against the cultured bacterial cells (such as aztreonam or any β-lactam molecule that are not hydrolyzed by a carbapenemase of the invention).

Finally, said candidate compound is positively selected at the end of the method if it reduces the number of CFUs, as compared with the number of CFUs found in the corresponding negative control cultures.

Thus, another object of the present invention consists of a method for the ex vivo screening of a candidate antibacterial substance which comprises the steps of:

a) performing a method for the in vitro screening of a antibacterial substances as disclosed in the present specification, with a candidate substance; and

b) assaying a candidate substance that has been positively selected at the end of step a) for its ex vivo antibacterial activity.

Inhibitor substances that have been positively selected at the end of any one of the screening methods that are previously described in the present specification may then be assayed for their in vivo antibacterial activity in combination with a β-lactam compound, in a further stage of their selection as a useful antibacterial active ingredient of a pharmaceutical composition.

As explained above, the compound is tested in combination with a β-lactam compound against bacterial cells expressing a carbapenemase of the invention.

Thus, any substance that has been shown to behave like an inhibitor of a carbapenemase, after positive selection at the end of any one of the screening methods that are disclosed previously in the present specification, may be further assayed for his in vivo antibacterial activity.

Consequently, any one of the screening methods that are described above may comprise a further step of assaying the combination of a positively selected inhibitor substance and a β-lactam substance for its in vivo antibacterial activity.

Usually, said further step consists of administering said combination to a mammal and then determining the antibacterial activity of said combination.

Mammals are preferably non human mammals, at least at the early stages of the assessment of the in vivo antibacterial effect of the combination tested. However, at further stages, human volunteers may be administered with said combination to confirm safety and pharmaceutical activity data previously obtained from non human mammals.

Non human mammals encompass rodents like mice, rats, rabbits, hamsters, guinea pigs. Non human mammals and also cats, dogs, pigs, calves, cows, sheeps, goats. Non human mammals also encompass primates like macaques and baboons.

Thus, another object of the present invention consists of a method for the in vivo screening of a candidate antibacterial substance which comprises the steps of:

a) performing a method for the in vitro screening of a antibacterial substances as disclosed in the present specification, with a candidate substance; and

b) assaying a candidate substance that has been positively selected at the end of step a) in combination with a β-lactam substance for its in vivo antibacterial activity.

Preferably, serial of doses containing increasing amounts of the inhibitor substance are prepared in view of determining the antibacterial effective dose of said inhibitor substance (when used in combination with a β-lactam compound) in a mammal subjected to a bacterial infection. Generally, the ED₅₀ dose is determined, which is the amount of the inhibitor substance that makes the combination effective against a bacterial strain expressing a carbapenemase of the invention in 50% of the animals tested. In some embodiments, the ED₅₀ value is determined for various distinct bacteria species, in order to assess the spectrum of the antibacterial activity.

In certain embodiments, it is made use of serial of doses of the inhibitor substance tested ranging from 1 ng to 10 mg per kilogram of body weight of the mammal that is administered therewith.

Several doses may comprise high amounts of said inhibitor substance, so as to assay for eventual toxic or lethal effects of said inhibitor substance and then determine the LD₅₀ value, which is the amount of said inhibitor substance that is lethal for 50% of the mammal that has been administered therewith.

β-lactam compound is used at the normal dose actually used in antibacterial treatment.

Illustratively, the daily amount of imipenem to be administered to an adult patient weighing 80 kg will typically ranges from 1 g to 4 g.

Illustratively, the daily amount of meropenem, ertapenem, faropenem, doripenem or panipenem to be administered to an adult patient weighing 80 kg will typically be of about 1-2 g.

According to the invention, the inhibitor substance in combination with a β-lactam compound forms an antibacterial composition.

The antibacterial composition to be assayed may be used alone under the form of a solid or a liquid composition.

When the antibacterial composition is used alone, the solid composition is usually a particulate composition of said antibacterial composition, under the form of a powder.

When the antibacterial composition is used alone, the liquid composition is usually a physiologically compatible saline buffer, like Ringer's solution or Hank's solution, in which said antibacterial composition is dissolved or suspended.

In other embodiments, said antibacterial composition is combined with one or more pharmaceutically acceptable excipients for preparing a pre-pharmaceutical composition that is further administered to a mammal for carrying out the in vivo assay.

Before in vivo administration to a mammal, the antibacterial composition selected through any one of the in vitro screening methods above may be formulated under the form of pre-pharmaceutical compositions. The pre-pharmaceutical compositions can include, depending on the formulation desired, pharmaceutically acceptable, usually sterile, non-toxic carriers or diluents, which are defined as vehicles commonly used to formulate pharmaceutical compositions for animal or human administration. The diluent is selected so as not to affect the biological activity of the combination. Examples of such diluents are distilled water, physiological saline, Ringer's solutions, dextrose solution, and Hank's solution. In addition, the test composition or formulation may also include other carriers, adjuvants, or non-toxic, non-therapeutic, non-immunogenic stabilizers and the like.

Compositions comprising such carriers can be formulated by well known conventional methods. These test compositions can be administered to the mammal at a suitable dose. Administration of the suitable compositions may be effected by different ways, e.g., by intravenous, intraperitoneal, subcutaneous, intramuscular, topical, intradermal, intranasal or intrabronchial administration. The dosage regimen will be determined by taking into account, notably, clinical factors. As is well known in the medical arts, dosages for any one mammal depends upon many factors, including the mammal's size, body surface area, age, the particular compound to be administered, sex, time and route of administration and general health. Administration of the suitable pre-pharmaceutical compositions may be effected by different ways, e.g., by intravenous, intraperitoneal, subcutaneous, intramuscular, topical or intradermal administration. If the regimen is a continuous infusion, it should also be in the range of 1 ng to 10 mg units per kilogram of body weight per minute, respectively. Progress can be monitored by periodic assessment. The pre-pharmaceutical compositions of the invention may be administered locally or systemically. Administration will generally be parenterally, e.g., intravenously. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, anti-oxidants, chelating agents, and inert gases and the like.

The antibacterial composition may be employed in powder or crystalline form, in liquid solution, or in suspension.

The injectable pre-pharmaceutical compositions may take such forms as suspensions, solutions, or emulsions in oily or aqueous vehicles, and may contain various formulating agents. Alternatively, the active ingredient may be in powder (lyophilized or non-lyophilized) form for reconstitution at the time of delivery with a suitable vehicle, such as sterile water. In injectable compositions, the carrier is typically comprised of sterile water, saline, or another injectable liquid, e.g., peanut oil for intramuscular injections. Also, various buffering agents, preservatives and the like can be included.

Topical applications may be formulated in carriers such as hydrophobic or hydrophilic base formulations to provide ointments, creams, lotions, in aqueous, oleaginous, or alcoholic liquids to form paints or in dry diluents to form powders.

Oral pre-pharmaceutical compositions may take such forms as tablets, capsules, oral suspensions and oral solutions. The oral compositions may utilize carriers such as conventional formulating agents and may include sustained release properties as well as rapid delivery forms.

In certain embodiments of the in vivo screening assay, the antibacterial composition is administered to a mammal which is the subject of a bacterial infection. For non human mammals, these animals have been injected with a composition containing bacteria prior to any administration of the inhibitor compound.

In certain other embodiments of the in vivo screening assay, non human animals are administered with the inhibitor compound to be tested prior to being injected with a composition containing bacteria.

Generally, non human mammals are injected with a number of bacterial cells expressing a carbapenemase of the invention cells ranging from 1×10² to 1×10¹² cells, including from 1×10⁶ to 1×10⁹ cells. In some embodiments, bacterial cells expressing a carbapenemase of the invention cells in an in vitro-generated dormant state are used for injection.

Generally, bacteria cells that are injected to the non human mammals are contained in a physiologically acceptable liquid solution, usually a saline solution like Ringer's solution or Hank's solution.

Generally, in the embodiment wherein the inhibitor compound to be tested is administered subsequently to bacterial inoculation, said inhibitor compound is administered form 1 hour to 96 hours after bacterial injection, including from 6 hours to 48 hours after bacterial injection.

Generally, in the embodiment wherein the inhibitor compound to be tested is administered prior to bacterial injection, said inhibitor compound is administered from 1 min to 3 hours prior to bacterial injection.

Generally, all animals are sacrificed at the end of the in vivo assay.

For determining the in vivo antibacterial activity of the inhibitor compound that is tested, blood or tissue samples of the tested animals are collected at determined time periods after administration of said inhibitor compound and bacteria counts are performed, using standard techniques, such as staining fixed slices of the collected tissue samples or plating the collected blood samples and counting the bacterial colonies formed.

Then, the values of the bacteria counts found for animals having been administered with increasing amounts of the inhibitor compound tested are compared with the value(s) of bacteria count(s) obtained from animals that have been injected with the same number of bacteria cells but which have not been administered with said inhibitor compound.

As already disclosed earlier in the present specification, various β-lactam candidate compounds have been assayed with the screening method of the invention and have been positively selected as compounds having a great potential value for treating individuals who have been infected by a bacterial strain expressing a carbapenemase of the invention.

Another object of the invention relates to an inhibitor of a carbapenemase of the invention in association with a β-lactam compound for an antibacterial treatment.

The invention also relates to an antibacterial composition containing an inhibitor of a carbapenemase of the invention and a β-lactam compound for an antibacterial treatment.

This invention also pertains to a method for treating individuals infected by gram negative bacteria expressing a carbapenemase of the invention comprising a step of administering to the said individuals an effective amount of an antibacterial composition of the invention.

Preferably, said antibacterial comprises one or more pharmaceutically acceptable excipient(s).

Such antibacterial compositions are under the form of dosage forms adapted for a daily administration of an amount of β-lactam of at least 1 mg and up to 10 g.

The effective amount of each component of antibacterial composition may be easily adapted by the one skilled in the art, depending notably on the age and of the weight individual to be treated.

The daily amount of each component of antibacterial composition may be administered to the patient through one or more uptakes, e.g. from one to six uptakes.

### Kits and Compositions of the Invention

The present invention also relates to compositions or kits for the screening of antibacterial substances.

In certain embodiments, said compositions or kits comprise a purified carbapenemase of the invention, preferably under the form of a recombinant protein.

In said compositions or said kits, said carbapenemase may be under a solid form or in a liquid form. Solid forms encompass powder of said carbapenemase under a lyophilized form. Liquid forms encompass standard liquid solutions known in the art to be suitable for protein long time storage.

Preferably, said carbapenemase is contained in a container such as a bottle, e.g. a plastic or a glass container. In certain embodiments, each container comprises an amount of said carbapenemase ranging from 1 ng to 10 mg, either in a solid or in a liquid form.

Further, said kits may comprise also one or more reagents, typically one or more substrate(s), necessary for assessing the enzyme activity of said carbapenemase.

Illustratively, if said kit comprises a container of carbapenemase, then said kit may also comprise a container comprising an appropriate amount of the substrate.

In certain embodiments, a kit according to the invention comprises one or more of each of the containers described above.

In another embodiment, said kits or compositions of the invention may also comprise a β-lactam compound for assessing the activity of the inhibitors selected by the screening methods of the invention.

Particularly, said β-lactam compound can be selected among the group of ticarcillin, piperacillin-tazobactam, imipenem, meropenem, ceftazidime and cefepime.

### Predictive Methods of the Invention

The inventors have shown that the carbapenemase DIM-1 is responsible for a resistance mechanism against compounds of the family of β-lactams, except the monobactam aztreonam.

Thus, a further object of the invention relates to a method for detecting or predicting a resistance mechanism of a microorganism against β-lactams comprising the step of assaying the presence or the expression of a gene encoding a carbapenemase of the invention in said microorganism.

The presence of said gene can be assayed by detecting the DNA sequence of a carbapenemase of the invention in the genome of the microorganism of interest or by detecting the expression of said gene, at mRNA or protein level in a sample containing said microorganism.

Several methods are well known in the art.

Said gene may for example be subjected to amplification by polymerase chain reaction (PCR), using specific oligonucleotide primers that enable amplification of a region in the nucleic acid of a carbapenemase of the invention. Said gene may be amplified, after which amplified sequences may be detected by hybridization with a suitable probe or by direct sequencing, or any other appropriate method known in the art.

In a particular embodiment of the invention, the presence of a gene encoding for a carbapenemase of the invention can be assayed using the pair of specific primers defined by the nucleic acid sequence SEQ ID NO:3 (TCTATTCAGCTTGTCTTCGC) for the sense primer and the nucleic acid sequence SEQ ID NO:4 (TGTTAGAGGCTGTCTCAGCC) for the antisense primer.

The expression of a gene encoding for a carbapenemase of the invention can be assayed by detecting the mRNA or protein encoded by said gene.

Methods for detecting mRNA are well known in the art. For example, the nucleic acid contained in the samples containing the microorganism of interest is first extracted according to standard methods, for example using lytic enzymes or chemical solutions or extracted by nucleic-acid-binding resins following the manufacturer's instructions. The extracted mRNA may be then detected by hybridization (e.g., Northern blot analysis).

Alternatively, the extracted mRNA may be subjected to coupled reverse transcription and amplification, such as reverse transcription and amplification by polymerase chain reaction (RT-PCR), using specific oligonucleotide primers that enable amplification of a region in the nucleic acid of a carbapenemase of the invention may be used. Quantitative or semi-quantitative RT-PCR is preferred. Real-time quantitative or semi-quantitative RT-PCR is particularly advantageous. Extracted mRNA may be reverse transcribed and amplified, after which amplified sequences may be detected by hybridization with a suitable probe or by direct sequencing, or any other appropriate method known in the art.

Other methods of Amplification include ligase chain reaction (LCR), transcription mediated amplification (TMA), strand displacement amplification (SDA) and nucleic acid sequence based amplification (NASBA).

Determination of the expression of a gene of interest can be easily assayed by detection of the protein encoded by said gene.

Such methods comprise contacting a sample susceptible of containing said gene (so, according to the invention, containing the microorganism of interest) with a binding partner capable of selectively interacting with the protein of interest present in the sample. The binding partner is generally an antibody that may be polyclonal or monoclonal, preferably monoclonal.

The presence of the said protein can be detected using standard electrophoretic and immunodiagnostic techniques, including immunoassays such as competition, direct reaction, or sandwich type assays. Such assays include, but are not limited to, Western blots; agglutination tests; enzyme-labelled and mediated immunoassays, such as ELISAs; biotin/avidin type assays; radioimmunoassays; immunoelectrophoresis; immunoprecipitation, immunocytochemistry, immunohistochemistry, etc. The reactions generally include revealing labels such as fluorescent, chemiluminescent, radioactive, enzymatic labels or dye molecules, or other methods for detecting the formation of a complex between the antigen and the antibody or antibodies reacted therewith.

More particularly, an ELISA method can be used, wherein the wells of a microtiter plate are coated with a set of antibodies against the proteins to be tested. A biological sample containing or suspected of containing the marker protein is then added to the coated wells.

After a period of incubation sufficient to allow the formation of antibody-antigen complexes, the plate(s) can be washed to remove unbound moieties and a detectably labelled secondary binding molecule added. The secondary binding molecule is allowed to react with any captured sample marker protein, the plate washed and the presence of the secondary binding molecule detected using methods well known in the art.

A further object of the invention relates to a method for predicting the response to an antibacterial treatment containing a β-lactam compound and an inhibitor of a carbapenemase of the invention in a patient, comprising the step of determining if the microorganism responsible for the infection in said patient expresses a carbapenemase of the invention.

An another further object of the invention relates to a method for predicting the response to an antibacterial treatment using aztreonam in a patient comprising the step of determining if the microorganism responsible for the infection in said patient expresses a carbapenemase of the invention.

The invention will be further illustrated by the following figures and examples. However, these examples and figures should not be interpreted in any way as limiting the scope of the present invention.

### EXAMPLE

Material & Methods

Bacterial strains. P. stutzeri clinical isolate 13 was identified with the API-20 NE system (BioMérieux, Marcy l'Etoile, France), and confirmed by rRNA sequencing. Escherichia coli TOP10 (In Vitrogen, Carlsbad, Calif.)was the host for cloning experiments (Poirel et al., 2001)

Susceptibility testing. Antibiotic-containing disks were used for routine antibiograms by the disk diffusion assay (Sanofi-Diagnostic Pasteur, Marnes-la-Coquette, France). The ESBL double-disk synergy test was performed with disks containing ceftazidime or cefepime and ticarcillin-clavulanic acid on Mueller-Hinton agar plates, and the results were interpreted as described previously (Poirel, Le Thomas et al., 2000). The carbapenemase detection was performed by using Etest carbapenem-containing strips (AB Biodisk, Solna, Sweden).

MICs were determined by an agar dilution technique with Mueller-Hinton agar (Sanofi-Diagnostic Pasteur) with an inoculum of 10⁴ CFU/ml, as described previously (Poirel, Le Thomas et al., 2000). All plates were incubated at 37° C. for 18 h at ambient atmosphere. MICs of β-lactams were determined alone or in combination with a fixed concentration of clavulanic acid (2 μg/ml), tazobactam (4 μg/ml), and sulbactam (4 μg/ml). MIC results were interpreted according to the guidelines of the National Committee for Clinical Laboratory Standards (NCCLS) (CLSI).

PCR and hybridization experiments. Total DNA of P. stutzeri 13 was extracted as described previously (Poirel et al., 1999). This DNA was used as a template in standard PCR conditions (Sambrook et al, 1989) with a series of primers designed for the detection of class B carbapenemase genes bla_{IMP}, bla_{VIM}, bla_{SPM}, and bla_{SI} (Corvec et al., 2008). Southern hybridizations were performed as described by Sambrook et al. (1989) using the ECL nonradioactive labelling and detection kit (GE Healthcare, Orsay, France).

Cloning experiments, recombinant plasmid analysis, and DNA sequencing. Total DNA of P. stutzeri 13 isolate was digested by XbaI restriction enzyme, ligated into the XbaI site of plasmid pBK-CMV and transformed in E. coli TOP10 reference strain, as described (Poirel et al., 2000a). Recombinant plasmids were selected onto Trypticase soy (TS) agar plates containing amoxicillin (50 μg/ml) and kanamycin (30 μg/ml). The cloned DNA fragments of several recombinant plasmids were sequenced on both strands with an Applied Biosystems sequencer (ABI 3100) (Applied Biosystems, Foster City, Calif.). The entire sequence provided in this study was made of sequences of several plasmids that contained overlapping cloned fragments. The nucleotide and deduced amino acid sequences were analyzed and compared to sequences available over the Internet at the National Center for Biotechnology Information website.

Genetic support. Transformation experiments were performed with P. stutzeri 13 DNA into P. aeruginosa PU21 recipient strain, as described (Rodriguez-Martinez et al. 2009) Plasmid DNA extraction from P. stutzeri 13 was attempted with the Qiagen plasmid DNA maxi kit (Qiagen, Courtaboeuf, France) and with the Kieser method (Poirel, Naas et al., 2000). To search for a chromosomal location of the carbapenemase gene, we used the endonuclease I-CeuI (New England Biolabs, Ozyme) (Riccio et al., 2005) which digests a 26-bp sequence in rrn genes for the 23S large-subunit rRNA and separated the fragments by pulsed-field gel electrophoresis, as described (Poirel et al. 2000b). Hybridization was performed with two different probes: a 1,504-bp PCR-generated probe specific for 16S and 23S rRNA genes (Poirel et al., 2000b)and a 688-bp probe specific for bla_{DIM-1} gene generated with internal primers DIM-1A (5′-TCTATTCAGCTTGTCTTCGC-3′, SEQ ID NO:3) and DIM-1B (5′-TGTTAGAGGCTGTCTCAGCC-3′, SEQ ID NO:4).

Carbapenemase purification and isoelectric focusing (IEF) analysis. Cultures of E. coli TOP10(pXD-1) were grown overnight at 37° C. in four liters of TS broth containing amoxicillin (100 μg/ml) and kanamycin (30 μg/ml). Carbapenemase was purified by ion-exchange chromatography. Briefly, the carbapenemase extract was sonicated, cleared by ultracentrifugation, treated with DNAse, and dialyzed against 20 mM diethanolamine buffer (pH 8.9). This extract was loaded on the Q-Sepharose column, and the carbapenemase-containing fractions were eluted with a linear 0 to 0.5 M NaCl gradient. The fractions containing the highest carbapenemase activity were again dialyzed against the same buffer mentioned above, and the same procedure repeated by eluting more slowly with a linear 0 to 0.2 M NaCl gradient. The purity of the enzyme was estimated by sodium dodecyl sulfate (SDS)-polyacrylamide gel electrophoresis analysis (Sambrook et al, 1989).

IEF analysis was performed with an ampholine polyacrylamide gel (pH 3.5 to 9.5), as described previously (Philippon et al., 1997) using a purified carbapenemase extract from a culture of E. coli DH10B(pXD-1). The focused carbapenemase were detected by overlaying the gel with 1 mM nitrocefin (Oxoid, Dardilly, France) in 100 mM phosphate buffer (pH 7.0).

The putative location of the signal peptide cleavage site has been determined by using software available on the SignalP 3.0 Server (http://www.cbs.dtu.dk/services/SignalP/).

Kinetic measurements. Purified carbapenemase was used for kinetic measurements performed at 30° C. with 100 mM sodium phosphate (pH 7.0) with an ULTROSPEC 2000 UV spectrophotometer (Amersham Pharmacia Biotech). Fifty percent inhibitory concentrations (IC₅₀s) were determined for clavulanic acid, tazobactam, sulbactam, cefoxitin, moxalactam and imipenem. Various concentrations of these inhibitors were preincubated with the purified enzyme for 3 min at 30° C. to determine the concentrations that reduced the hydrolysis rate of 100 μM benzylpenicillin by 50%.

The specific activity of the purified carbapenemase from E. coli DH10B(pXD-1) was obtained as described previously (Poirel et al., 1999). One unit of enzyme activity was defined as the activity which hydrolyzed 1 μmol of imipenem per min per mg of protein. The total protein content was measured with the DC Protein assay kit (Bio-Rad, Ivry-sur-Seine, France).

Nucleotide sequence accession number. The nucleotide sequences data reported in this work have been deposited in the GenBank nucleotide database under accession no. DQ089809.

Results

Properties of P. stutzeri isolate 13. This strain was isolated in June 2007 at the VU Medical Center, Amsterdam, The Netherlands, from a pus of a 55-year-old man hospitalized for a surgery of the tibia after development of a chronic osteomyelitis. That patient did not have any history of recent travel or hospitalization elsewhere. P. stutzeri 13 was resistant to ticarcillin, piperacillin, piperacillin-tazobactam, and imipenem, had reduced susceptibility to ceftazidime, cefepime, cefpirome and was fully susceptible to aztreonam. Double-disk synergy testing was negative with clavulanate-ceftazidime and clavulanate-imipenem combinations, but was positive with the carbapenem-containing E-test (MIC of IMP at 64 μg/m1 vs MIC of IMP/EDTA at 2 μg/ml). P. stutzeri 13 was also resistant to gentamicin, tobramycin, fluoroquinolones, rifampicin, chloramphenicol and tetracycline, and remained susceptible to amikacin, netilmicin, and colistin.

Cloning and sequencing of the carbapenemase gene. Preliminary attempts to detect by PCR carbapenemase encoding genes failed. Using total DNA of P. stutzeri 13 as a template in cloning experiments, several recombinant plasmids including pXD-1 were obtained. Sequence analysis of a ca. 10-kb cloned fragment of pXD-1 revealed a 756-bp long open reading frame (ORF) encoding a 251-amino-acid preprotein corresponding to an Ambler class B carbapenemase designated DIM-1 (for Dutch IMipenemase). It possessed the conserved motifs characteristic of class B enzymes (Galleni et al., 2001) including the consensus zinc binding motif HXHXD (residues 116 to 120), together with His196, Cys221, and His293 according to the BBL nomenclature (Galleni et al., 2001) The G+C content of bla_{DIM-1} was 43.5%, a value which differs significantly from the G+C content of the P. stutzeri genome being 63% accoring to the Genbank database (n°NC_{—}009434). DIM-1 was distantly related to other class B carbapenemases. Indeed, the highest percentages of amino acid identity were 52% with GIM-1 (Genbank accession number n°CAF05908), 49% with a putative carbapenemase identified in-silico in the genome of Shewanella denitrificans (Genbank NC_{—}007954), and 48% with KHM-1 (Sekiguchi et al. 2008). The carbapenemase DIM-1 shared 45% identity with the widespread IMP-type enzymes, and only 30% with the VIM-type enzymes. A phylogenetic tree performed with other carbapenemases indicates that DIM-1 clusters together with the S. denitrificans carbapenemase, and with GIM-1 to a lesser extend.

β-Lactam susceptibility. MICs of β-lactam for P. stutzeri 13 and for E. coli DH10B(pXD-1) indicated the expression of a carbapenemase that hydrolysed expanded-spectrum cephalosporins (including cephamycins) together with carbapenems, that conferred reduced susceptibility to imipenem and meropenem, and that paradoxally spared aztreonam. Addition of carbapenemase inhibitors such as clavulanic acid or tazobactam did not restore any susceptibility to penicillins.

Biochemical properties of DIM-1. IEF analysis showed that P. stutzeri 13 and E. coli TOP10(pXD-1) had carbapenemase activities with a pI value of 6.2, corresponding to that of DIM-1. The specific activity of the purified carbapenemase DIM-1 was 21 U.mg of protein⁻¹. Its overall recovery was 80% with a 45-fold purification. The purity of the enzyme was estimated to be more than 95% according to SDS gel electrophoresis analysis. Kinetic parameters of DIM-1 showed its broad-spectrum activity against most β-lactams, including oxyimino-cephalosporins, cephamycins, and carbapenems but excluding aztreonam. Analysis of the relative hydrolysis rates of DIM-1 showed that cefotaxime was hydrolyzed at a similar level as benzylpenicillin, and cefoxitin was also a good substrate. Ceftazidime was well hydrolyzed, with a Km value of 50 μM reflecting a relatively good affinity of DIM-1 for ceftazidime, as commonly observed with many carbapenemases (Sekiguchi et al. 2008, Poirel et al. 2000b). IC₅₀ determinations performed with benzylpenicillin as a substrate showed that DIM-1 activity was inhibited by EDTA (175 mM).

Genetic environment of bla_{DIM-1}. Sequence analysis of recombinant plasmid pXD-1 harboring the bla_{DIM-1} gene revealed that it was as a form of a gene cassette, which was inserted at the attI1 recombination site. Analysis of the 5′-end sequence of the integron showed that the P_{C} promoter sequences were located in the structural integrase gene but no secondary promoter P₂ was identified (Levesque et al., 1994). Thus, the gene cassettes located in that integron are under the control of weak promoter sequences.

The dim-1 gene cassette possessed imperfect core (GTTAGAG, SEQ ID NO:5) and inverse core (CGCTAAC, SEQ ID NO:6) sites, that latter being located inside the bla_{DIM-1} coding sequence (23 bp from the 3′-end of the gene), and the length of its 59-be sequence was only 31 bp. A second gene cassette was identified, containing the aadB gene encoding resistance to aminoglyscosides. The third gene cassette contained the qacH gene encoding resistance to disinfectants. Inside the qacH gene cassette, the ISKpn4 insertion sequence was identified that had targeted the 59-be, as previously noticed with other members of the IS1111 family that target preferentially the gene cassette 59-bes (Post et al., 2009).

Analysis of the right extremity of this integron showed that the 3′-conserved segment made of the qacEΔ1 and sul1 genes usually identified were absent, but the tniC gene identified in defective derivatives of Tn402-like transposable elements was present. The tniA allele of the Tn402-tni module was flanked by the IRt extremity of the transposon.

Genetic support of the carbapenemase determinant. Mating-out assays as well as electro-transformation experiments did not allow transferring the carbapenemase encoding gene either to P. aeruginosa PU21 or E. coli TOP10 recipients strains. However, analysis of plasmid content of P. stutzeri 13 identified a single plasmid of ca. 70-kb in size, that harboured the bla_{DIM-1} gene as confirmed by Southern hybridization.

### REFERENCES

Throughout this application, various references describe the state of the art to which this invention pertains. The disclosures of these references are hereby incorporated by reference into the present disclosure.
Chang A C, Nunberg J H, Kaufman R J, Erlich H A, Schimke R T, Cohen S N. Nature. 1978 Oct. 19;275(5681):617-24.
Chevray, Nathans, Proc. Natl. Acad. Sci. USA, 89: 5789-5793 (1991)
Chien et al., Proc. Natl. Acad. Sci. USA, 88: 9578-9582 (1991)
Clinical and Laboratory Standards Institute. 2008. Performance standards for antimicrobial susceptibility testing. 18th informational supplement. Clinical and Laboratory standards, Wayne, Pa.
Corvec S, Poirel L, Espaze E, Giraudeau C, Drugeon H, Nordmann P. 2008 J. Hosp. Infect. 68 :73-82.
DeBoer L W, Rude R E, Kloner R A, Ingwall J S, Maroko P R, Davis M A, Braunwald E. Proc Natl Acad Sci USA. 1983 September;80(18):5784-8.
Fields and Song, 1989. Nature (London), 340: 245-246.
Galleni M, Lamotte-Brasseur J, Rossolini G M, Spencer J, Dideberg O, Frere J M, Metallo-b-lactamase working group. 2001 Antimicrob Agents Chemother 45, 660-663.
Goeddel D V, Heyneker H L, Hozumi T, Arentzen R, Itakura K, Yansura D G, Ross M J, Miozzari G, Crea R, Seeburg P H. Nature. 1979 Oct. 18;281(5732):544-8.
Goeddel D V, Shepard H M, Yelverton E, Leung D, Crea R, Sloma A, Pestka S. Nucleic Acids Res. 1980 Sep. 25;8(18):4057-74.
Levesque C, Brassard S, Lapointe J, Roy P H. 1994. Gene 142, 49-54.
Merrifield, J. Am. Chem. Soc. 1963. 85: 2149-2154
Philippon L N, Naas T, Bouthors A T, Barakett V, Nordmann P. 1997. Antimicrob Agents Chemother. 41, 2188-2135.
Poirel L, Girlich D, Naas T, Nordmann P. 2001. Antimicrob. Agents Chemother. 45; 447-453.
Poirel L, Guibert M, Girlich D, Naas T, Nordmann P. 1999. Antimicrob Agents Chemother 43, 769-776.
Poirel, L., I. Le Thomas, T. Naas, A. Karim, and P. Nordmann. 2000 a. Antimicrob. Agents Chemother. 44:622-632.
Poirel L, Naas T, Guibert M, Chaibi E B, Labia R, Nordmann P. 1999. Antimicrob Agents Chemother. 43:573-581.
Poirel L., T. Naas, D. Nicolas, L. Collet, S. Bellais, J. D. Cavallo, and P. Nordmann. 2000b. Antimicrob. Agents Chemother. 441891-897.
Post V, Halll H M. 2009. FEMS Microbiol Lett 290, 182-187.
Riccio, M. L., L. Pallecchi, J. D. Docquier, S. Cresti, M. R. Catania, L. Pagani, C. Lagatolla, G. Cornaglia, R. Fontana and G. M. Rossolini. 2005. Antimicrob. Agents Chemother. 49:104-110.
Rodriguez-Martinez J M, Poirel L, Nordmann P. Antimcrob Agents Chemother. 53:1766-1771.
Sagiguchi J, Morita K, Kitao T, Watanabe N, Okazaki M, Miyoshi-Akiyama T, Kanamori M, Kirikae T. 2008. Antimicrob Agents Chemother. 52, 4194-4197.
Sambrook J, Fritsch E F, Maniatis T. 1989. Molecular cloning: a laboratory manual, 2^{nd} ed. Cold Spring Harbor Laboratory Press, Cold Spring Harbor, N Y
Stewart et al., Solid-Phase Peptide Synthesis (W. H. Freeman Co.: San Francisco, Calif., 1969);

1. A purified carbapenemase comprising
i) the amino acid sequence of SEQ ID NO:1 or
ii) an amino acid sequence having at least 80% amino acid sequence identity with the amino acid sequence of SED ID NO:1.

2. A nucleic acid sequence encoding a carbapenemase according to claim 1.

3. A nucleic acid sequence according to claim 2, wherein said nucleic acid sequence comprises SEQ ID NO:2.

4. A method for screening a candidate substance for antibacterial activity, comprising the step of
determining the ability of the candidate substance to inhibit activity of a purified carbapenemase according to claim 1.

5. A method according to claim 4, wherein said step of determining comprises the steps of:
(i) providing a composition comprising a carbapenemase according to claim 1 and a substrate thereof,
(ii) adding the candidate substance to be tested to the composition provided at step (i), thereby providing a test composition;
(iii) comparing the activity of said carbapenemase in the test composition with the activity of said carbapenemase in the absence of said candidate substance; and
(iv) if said candidate substance that inhibits the catalytic activity of said carbapenemase, then concluding that said candidate substance has antibacterial activity.

6. A method for screening a candidate substance for antibacterial activity, comprising the step of:
assaying said candidate substance for its ability to bind to a carbapenemase according to claim 1.

7. The method of claim 6, wherein said step of assaying includes the steps of:
(i) contacting said candidate substance with a carbapenemase according to claim 1;
(ii) detecting complexes formed between said carbapenemase and said candidate substance, and
(iii) if complexes are formed between said carbapenemase and candidate substance, then concluding that said candidate substance has antibacterial activity.

8. A method for detecting or predicting a resistance of a microorganism against β-lactams comprising the step of
assaying the presence or the expression of a gene encoding a carbapenemase according to claim 1 in said microorganism, and
when the presence or the expression said gene encoding said carbapenemase is detected in said microorganism, concluding that said microorganism is resistant to β-lactams.

9. A method for predicting the response of a patient with an infection to an antibacterial treatment containing a β-lactam compound and an inhibitor of a carbapenemase according to claim 1, comprising the steps of
determining if the microorganism responsible for the infection in said patient expresses said carbapenemase; and
if said microorganism does express said carbapenemase, concluding that said patient will respond positively to said antibacterial treatment.

10. (canceled)

11. The method of claim 9, wherein said antibacterial treatment comprises aztreonam.
